(19) United States
(12) Patent Application Publication
(10) Pub. No.: US 2011/0105909 A1
(43) Pub. Date: May 5, 2011

(54) NEAR-INFRARED LIGHT BRAIN COMPUTER INTERFACE VISION DRIVEN CONTROL DEVICE AND ITS METHOD

(75) Inventors: Chia-Wei SUN, Taipei County (TW); Po-Lei LEE, Taipei City (TW)

(73) Assignee: NATIONAL YANG-MING UNIVERSITY, TAIPEI CITY (TW)

(21) Appl. No.: 12/628,815

(22) Filed: Dec. 1, 2009

(30) Foreign Application Priority Data
Oct. 30, 2009 (TW) 098136959

(51) Int. Cl. A61B 6/00

(52) U.S. Cl. 600/473

(57) ABSTRACT

A near-infrared light brain computer interface vision driven control device and its method are applied for measuring a near-infrared light brain signal generated during a user with a sight ability to feel an optical image data so as to use the measured near-infrared light brain signal as a signal source to control peripherals. The control device at least includes an optical image display unit, a near-infrared light measuring unit, a signal analysis processing unit, and a controlled unit.

### FIELD OF THE INVENTION

The present invention is related to a near-infrared light brain signal vision driven control device and its method, and more particularly to a brain computer interface device and its method by measuring a near-infrared light brain signal generated during a user with a sight ability to feel an optical image data and thus outputting a control signal.

### BACKGROUND OF THE INVENTION

The “brain computer interface (BCI)” is applied for requesting the user to perform a specific task so that a specific brain signal pattern is generated from the user, and the brain signal pattern would be measured and identified as a communication channel with external devices. Through this interface, the purposes for some patients to communicate with the outside, transmit message, independently act and self-care could be achieved by using only their brain signals without their peripheral nerves and muscles.

Nowadays, the brain computer interface is used by electroencephalogram (EEG) measurement as a communication signal between the user and the outside. The EEG based brain computer interface includes a non-invasive type and an invasive type. The brain computer interface of the non-invasive type is used for measuring brain electrical signals on the scalp as control signals of the BCI through analyzing amplitude changes in frequency of the brain signal or waveform changes of brain signal on time domain. Further, the brain computer interface of the invasive type is applied for direct-invasively measuring neural activating signal in the cerebral cortex by using subdural EEG or single-unit recording. However, no matter what is applied the non-invasive type or the invasive type, it still has restrictive use and shortcomings. Firstly, the measured brain signals are very weak (as a 10⁻⁶ Volts level) as compared with the noise in nature life (as a 10⁻³ Volts level), so that the measured brain signals are very easily interfered by external electromagnetic noise, thereby causing signal unreliable. Secondly, in the brain electrode of the invasive type, a signal processing and control chip must be implanted in the cerebral cortex. This implant surgery not only is dangerous and expensive, but also causes the risk of brain damage in the patient during the surgery. Thirdly, in the brain signal electrode of the non-invasive type, the conductive adhesive is smeared over the scalp to assist the brain signal transduction and data acquisition. However, under the situation of long-time using the conductive adhesive, it could cause discomfortable for the patients, such as allergy in localized scalp.

Therefore, it is a current problem for the engineering to be solved to develop a brain computer interface without contacting patient's skin to be free from electromagnetic interference and with reducing costs.

### SUMMARY OF THE INVENTION

Since the current brain computer interface uses a measurement analysis method in the EEG signals, it is easily influenced by external electromagnetic interference or the noise of the electromyograms (EMG) and the signals of the other area on cerebral cortex.

Accordingly, it is a main purpose of the present invention to provide a visual evoked near-infrared light based brain computer interface with non-invasive, high temporal resolution, no electromagnetic interference and non-contacting with skin.

In order to achieve the above purposes, the present invention discloses a near-infrared light brain signal vision driven control device, at least comprising:

an optical image display unit having multi-frequency flashing signals, each of the flashing signals is different;

a near-infrared light measuring unit for transmitting and receiving near-infrared light echo signals with different wavelengths to measure near-infrared light brain signals generated by a user;

a signal analysis processing unit for analyzing and calculating correlations between each of the flashing signals and the measured near-infrared light brain signals in a frequency domain or in a time domain to determine a flashing signal having a maximum correlation with the near-infrared light brain signals and output a corresponding control signal; and

a controlled unit receiving the control signal of the signal analysis processing unit and performing an action according to the control signal.

In order to achieve the above purpose, the present invention discloses a near-infrared light brain signal vision driven control method for measuring a near-infrared light brain signal generated during a user with a sight ability to feel an optical image data and outputting a control signal, the method at least comprising the steps of:

(A) displaying optical image data with different flashing frequencies;

(B) measuring near-infrared light brain signals of the user;

(C) determining a flashing signal having a maximum correlation with the near-infrared light brain signals; and

(D) outputting a control signal corresponding to the flashing signal having a maximum correlation with the near-infrared light brain signals

The details and the embodiments in the present invention are set forth in the following detailed description taken in conjunction with the accompanying drawings

### BRIEF DESCRIPTION OF THE DRAWINGS

FIG. 1 is a schematic view of conventional brain function areas.

FIG. 2 is a schematic view showing a circuit block diagram according to the present invention.

FIG. 3 is a schematic view showing a block diagram according to the embodiment of the present invention.

FIG. 4 is a flow chart showing a method according to the present invention.

### DETAILED DESCRIPTION OF THE PREFERRED EMBODIMENTS

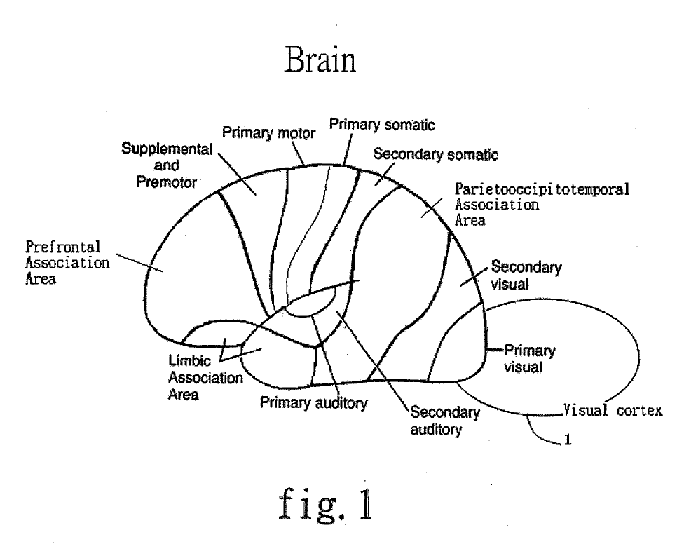

There are many functional areas in human brain, as shown in FIG. 1. When eyes receive external visual stimulation, activation of nerve cells is generated in a corresponding brain visual cortex 1. The activation of nerve causes nerve of the brain visual cortex 1 to produce electric signals, and thus generates electric and magnetic fields change and local blood flow changes. The electric and magnetic electromagnetic fields change would be detected by using the EEG or the magnetoencephalography (MEG), and blood flow changes would be measured by using the laser Doppler fluxmetry (LDF) and the functional magnetic resonance imaging (fMRI). In the present invention, when a user watches a light source with a specific frequency, a corresponding near-infrared light brain signal with the same frequency of the flashing signal is generated in the brain visual cortex 1. Thus, it could be determined which light source the user is watching by comparing the measured frequency of the near-infrared light brain signal in the visual cortex 1 with the frequency of the flashing signal, as a basis to be a control signal to control devices.

That is to say, if the eyes are stimulated by a predetermined flashing light, the information of light signal is transmitted to the brain visual cortex 1 through optic nerves, and the blood flow in a local area is increased. Accordingly, it would be provided a visual evoked near-infrared light based brain computer interface by applying this principle.

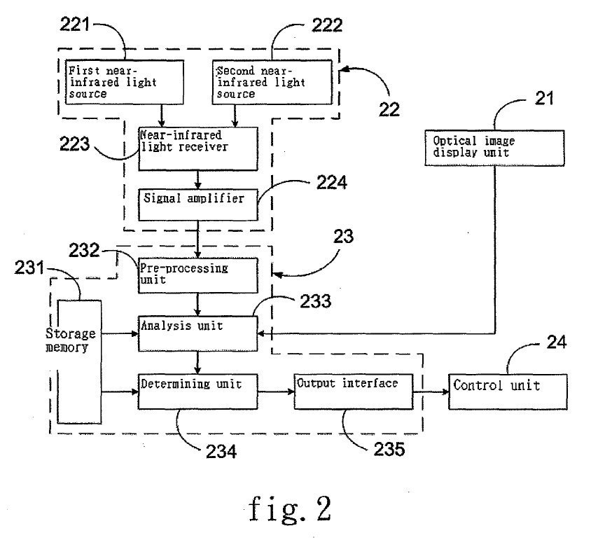

Please refer to FIG. 2, which is a circuit block diagram according to the present invention.

The present near-infrared light brain signal vision driven control device, at least includes an optical image display unit 21, a near-infrared light measuring unit 22, a signal analysis processing unit 23 and a controlled unit 24.

The optical image display unit 21 has multi-timing flashing signals. The respective flashing signals are different and have respective different flashing frequencies.

The near-infrared light measuring unit 22 is used for transmitting and receiving near-infrared light echo signals with different wavelengths to measure near-infrared light brain signals generated by a user. The near-infrared light measuring unit 22 at least includes a first near-infrared light source 221 having a near-infrared light source lower than a wavelength of 800 nm, a second near-infrared light source 222 having a near-infrared light source higher than a wavelength of 800 nm, a near-infrared light source receiver 223 receiving the near-infrared light echo signals having two different wavelengths of near-infrared light, and a signal amplifier 224 amplifying the near-infrared light backscattering signals to output it.

The signal analysis processing unit 23 is used for analyzing and calculating correlations between the respective flashing signals and the measured near-infrared light brain signals in a frequency domain or in a time domain, and determining a flashing signal having a maximum correlation with the near-infrared light brain signals and output a corresponding control signal. Further, the signal analysis processing unit 23 at least includes a storage memory 231 for storing a default threshold, each of the flashing signals and multi-command corresponding to the flashing signals, a pre-processing unit 232 for filtering out noise from the amplified near-infrared light backscattering signals, a analysis unit 233 for analyzing and calculating the correlations between each of the flashing signals and the near-infrared light brain signals in the frequency domain or in the time domain, a determining unit 234 for determining the flashing signal having a maximum correlation with the near-infrared light brain signal by comparing the correlations calculated by the analysis unit and transmitting a control signal corresponding to the flashing signal having a maximum correlation, and an output interface 235 for receiving the corresponding control signal and outputting it.

The control unit 24 is used for receiving the control signal of the signal analysis processing unit 23 and performing an action according to the control signal.

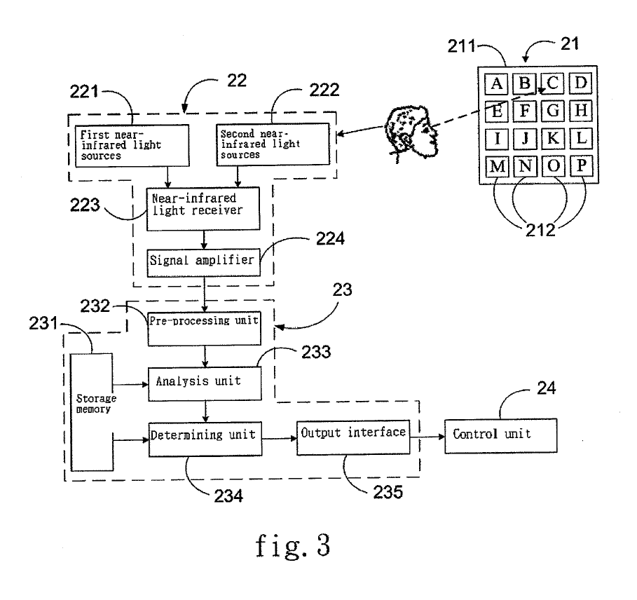

Please refer to FIG. 3, which is a block diagram according to the scheme of the present invention.

In the present scheme, the optical image display unit 21 is a liquid crystal display (LCD) connected to the signal analysis processing unit 23 and includes a LCD screen for displaying an optical image data 212. The optical image data 212 includes the characteristics of multi-frequency vision excitation. According to the present scheme, the options A to P include various flashing frequencies. However, it should be noted that the optical image data 212 is not limited to the frequency distinction in the scheme and any coding methods for discriminating different option from the screen could be included in protecting scope of the present device. The near-infrared light measuring unit 22 in the present scheme have near-infrared light sources 221, 222 with two different wavelength (i.e. respective wavelengths of 650 nm˜800 nm and 800 nm˜900 nm in the present scheme). The two wavelengths light provide different detecting abilities from the concentrations of oxy-hemoglobin and deoxy-hemoglobin in the brain, so as to respectively detect the concentrations of oxy-hemoglobin and deoxy-hemoglobin in local area of the brain. Signals are received by the near-infrared light source receiver 223 and amplified by the signal amplifier 224, and data measured by the near-infrared light measuring unit 22 is outputted into the signal analysis processing unit 23. After filtering out noise from the amplified near-infrared light backscattering signals in the pre-processing unit 232, the analysis unit 233 performs a spectrum analysis or a time-frequency analysis and calculates the correlations between the related flashing signals and the near-infrared light brain signals in the frequency domain or in the time domain according to the predetermined flashing signal in the optical image data 212. The determining unit 234 determines the flashing signal having a maximum correlation with the measured near-infrared light brain signal by comparing the correlations calculated by the analysis unit 233 and then compares the maximum correlation with the default threshold in the storage memory 231. If the maximum correlation is less than the default threshold, the near-infrared light measuring unit 22 re-receives the brain signal and then the analysis unit 233 analyzes the correlation to re-determine. Otherwise, the determining unit 234 defines the flashing signal having a maximum correlation as a selected signal to send a corresponding control signal and the corresponding control signal would be outputted from the output interface 235. Of course, it could be understood for those skilled in the art that the pre-processing unit 232 is not absolutely necessary and the analysis unit 233 and the determining unit 234 could be combined.

In the present scheme, the respective flashing signals recorded in the storage memory and a multi-command corresponding to the flashing signals are shown as following table:

| | TABLE | | | |
|---|---|---|---|---|
| | Flashing signal | | | |
| | 0.1 Hz | 0.2 Hz | 0.3 Hz | 0.4 Hz |
| Corresponding | Display A | Display B | Display C | Display D |
| instruction | | | | |
| | Flashing signal | | | |
| | 0.5 Hz | 0.6 Hz | 0.7 Hz | 0.8 Hz |
| Corresponding | Display E | Display F | Display G | Display H |
| instruction | | | | |
| | Flashing signal | | | |
| | 0.9 Hz | 1.0 Hz | 1.1 Hz | 1.2 Hz |
| Corresponding | Display I | Display J | Display K | Display L |
| instruction | | | | |
| | Flashing signal | | | |
| | 1.3 Hz | 1.4 Hz | 1.5 Hz | 1.6 Hz |
| Corresponding | Display M | Display N | Display O | Display P |
| instruction | | | | |

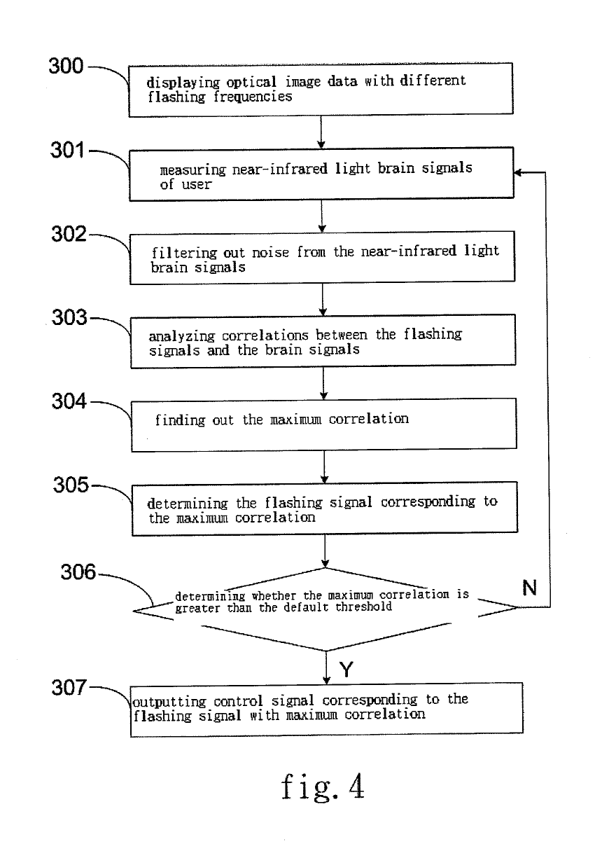

The following brief description discloses the implemented process according to the scheme of the present invention, as shown in FIG. 4. The present near-infrared light brain signal vision driven control method is as follows.

Step 300 is to display optical image data 212 with different flashing frequencies.

Step 301 is to measure near-infrared light brain signals of the user by the near-infrared light measuring unit 22. If the user watches the block of letter “C”, the measured value of the near-infrared light brain signal is 0.3 Hz.

Step 302 is to filter out frequency lower than 0.01 Hz or greater than 2 Hz from the near-infrared light brain signals by the pre-processing unit 232. Since the frequency displayed in the above optical image data 212 is between 0.1 Hz and 1.6 Hz, the frequency lower than 0.01 Hz and greater than 2 Hz in the near-infrared light brain signals should be filtered out.

Step 303 is to analyze and calculate correlations between the near-infrared light brain signals and the respective flashing signals. In the present embodiment, the analysis unit 233 would find out the correlation between the near-infrared light brain signal with a frequency nearest to 0.3 Hz and the respective flashing signals in the mentioned table.

Step 304 is to compare the correlations analyzed in the step 303 and assume that the present maximum correlation is 100%

Step 305 is to find out the flashing signal corresponding to the maximum correlation by the determining unit 234 to be defined as the selected frequency for the user. In the present scheme, the flashing frequency in the flashing signal having a maximum correlation with the near-infrared light brain signals is 0.3 Hz, and the corresponding control signal is the instruction “Display C” in the table.

Step 306 is to compare the maximum correlation with the default threshold. In the present embodiment, the default threshold is supposed to be 90%. Since the maximum correlation is greater than the default threshold, the next step is continued, otherwise, Step 301 to Step 306 are repeated.

Step 307 is to output a control signal corresponding to the flashing signal with the maximum correlation.

In conclusion, the present invention provides a near-infrared light brain signal vision driven control device and its method applied for measuring the near-infrared light brain signals for the user to analyze and control, and thus it would be directly operated without adjusting for the relationship between the detector and the user before operating. Accordingly, the present invention provides an easy operation so that the application for the disabled person to the near-infrared light brain signal control method would be effectively.

While the invention has been described in terms of what are presently considered to be the most practical and preferred scheme, it is to be understood that the invention need not to be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A near-infrared light brain signal vision driven control device, at least comprising:
an optical image display unit having multi-frequency flashing signals, each of the flashing signals is different;
a near-infrared light measuring unit for transmitting and receiving near-infrared light backscattering signals with different wavelengths to measure near-infrared light brain signals generated by a user;
a signal analysis processing unit for analyzing and calculating correlations between each of the flashing signals and the measured near-infrared light brain signals in a frequency domain or in a time domain to determine a flashing signal having a maximum correlation with the near-infrared light brain signals and output a corresponding control signal; and
a control unit receiving the control signal of the signal analysis processing unit and performing an action according to the control signal.

2. The near-infrared light brain signal vision driven control device of claim 1, wherein the flashing signals have respective different flashing frequencies.

3. The near-infrared light brain signal vision driven control device of claim 1, wherein the near-infrared light measuring unit at least comprises:
a first near-infrared light source having a near-infrared light source lower than a wavelength of 800 nm;
a second near-infrared light source having a near-infrared light source higher than a wavelength of 800 nm;
a near-infrared light source receiver receiving the near-infrared light echo signals having two different wavelengths of near-infrared light; and
a signal amplifier amplifying the near-infrared light echo signals to output.

4. The near-infrared light brain signal vision driven control device of claim 1, wherein the signal analysis processing unit at least comprises:
a storage memory for storing a default threshold, each of the flashing signals and a plurality of instructions corresponding to the flashing signals;
a pre-processing unit filtering out noise from the amplified near-infrared light echo signals;
a analysis unit analyzing and calculating the correlations between each of the flashing signals and the near-infrared light brain signals in the frequency domain or in the time domain;
a determining unit determining the flashing signal having a maximum correlation with the near-infrared light brain signal by comparing the correlations calculated by the analysis unit and transmitting a control signal corresponding to the flashing signal having a maximum correlation; and
an output interface receiving the corresponding control signal and outputting it.

5. A near-infrared light brain signal vision driven control method for measuring a near-infrared light brain signal generated during a user with a sight ability to feel an optical image data so as to output a control signal, the method at least comprising the steps of:
(A) displaying optical image data with different flashing frequencies;
(B) measuring near-infrared light brain signals of the user;
(C) determining a flashing signal having a maximum correlation with the near-infrared light brain signals; and
(D) outputting a control signal corresponding to the flashing signal having a maximum correlation with the near-infrared light brain signals

6. The near-infrared light brain signal vision driven control method of claim 5, wherein the step (C) further includes the steps of:
(C1) analyzing the correlation between the flashing signal and the near-infrared light brain signals;
(C2) finding out the maximum correlation; and
(C3) determining the flashing signal corresponding to the maximum correlation.

7. The near-infrared light brain signal vision driven control method of claim 5, further comprising a step of pre-defining a default threshold comparing with the maximum correlation, and further comprising steps between the step (C) and step (D) of:
(a) determining whether the maximum correlation is greater than the default threshold;
(b) outputting a control signal if the maximum correlation is greater than the default threshold; and
(c) proceeding the step (3) and (4) if the maximum correlation is less than the default threshold;

8. A near-infrared light brain signal vision driven control method, characterized by using a near-infrared light to measure a brain functional area for a user to obtain a near-infrared light brain signal and using the obtained near-infrared light brain signal as a signal source to control peripherals.

9. The near-infrared light brain signal vision driven control method of claim 8, wherein the brain functional area is a brain visual cortex.
